(19) United States
(12) Patent Application Publication
(10) Pub. No.: US 2018/0304072 A1
(43) Pub. Date: Oct. 25, 2018

(54) PERCUTANEOUS MULTI-PRONGED LEAD

(71) Applicant: OHIO STATE INNOVATION FOUNDATION, Columbus, OH (US)

(72) Inventors: Ali REZAI, Morgantown, WV (US); Milind DEOGAONKAR, Westerville, OH (US)

(21) Appl. No.: 15/779,319

(22) PCT Filed: Nov. 25, 2016
(86) PCT No.: PCT/US2016/063757

§ 371 Date: May 25, 2018

Related U.S. Application Data
(60) Provisional application No. 62/260,024, filed on Nov. 25, 2015.

(51) Int. Cl. A61N 1/05

(52) U.S. Cl. CPC A61N 1/0558; A61N 1/0504

(57) ABSTRACT

A percutaneous, multi-pronged lead includes a lead body having a multi-pronged distal end portion. At least one of the prongs has an electrode connected thereto. Each of the prongs is configured to transition from a stored configuration into a deployed configuration such that the prongs at least partially wrap around a target tissue.

### RELATED APPLICATION

This application claims the benefit of U.S. Provisional Patent Application Ser. No. 62/260,024, filed Nov. 25, 2015, the entirety of which is hereby incorporated by reference for all purposes.

### TECHNICAL FIELD

The present disclosure relates generally to devices and methods for neuromodulation and, more particularly, to a percutaneous, multi-pronged lead and related methods for improved targeting and stimulation of nervous tissue.

### BACKGROUND

Electrical leads are commonly used to stimulate nervous tissue, such as peripheral nerve ganglia. To properly apply stimulation, however, one or more electrodes comprising the lead must be directly attached to the nervous tissue as such leads are incapable of grasping the nervous tissue without the use of a securing means (e.g., sutures). Direct attachment typically requires a complex surgical operation that involves forming a large incision in a patient.

### SUMMARY

The present disclosure relates generally to devices and methods for neuromodulation and, more particularly, to a percutaneous, multi-pronged lead and related methods for improved targeting and stimulation of nervous tissue.

One aspect of the present disclosure can relate to a percutaneous, multi-pronged lead that includes a lead body having a multi-pronged distal end portion. At least one of the prongs can have an electrode connected thereto. Each of the prongs can be configured to transition from a stored configuration into a deployed configuration such that the prongs at least partially wrap around a target tissue.

Another aspect of the present disclosure can relate to a percutaneous, multi-pronged lead consisting of a lead body having a multi-pronged distal end portion. At least one of the prongs can have an electrode connected thereto. Each of the prongs can be configured to transition from a stored configuration into a deployed configuration such that the prongs at least partially wrap around a target tissue.

Another aspect of the present disclosure can relate to a method for stimulating a target tissue in a subject. One step of the method can include providing a multi-pronged lead including a lead body with a multi-pronged distal end portion. At least one of the prongs can have an electrode connected thereto. The lead body can include a main body portion extending between the distal end portion and a proximal end portion. The lead can be placed in a deployment device so that the prongs obtain a stored configuration in which the prongs are arranged substantially parallel to the main body portion of the lead. A distal end portion of the deployment device can be positioned adjacent the target tissue. The distal end portion of the lead body can be deployed from the deployment device so that the prongs transition from the stored configuration to a deployed configuration and thereby at least partially wrap around the target tissue. An electrical signal can be delivered to the at least one electrode to stimulate the target tissue.

Another aspect of the present disclosure can relate to a method for stimulating a target tissue in a subject. One step of the method can include providing a multi-pronged lead including a lead body with a multi-pronged distal end portion. At least one of the prongs can have an electrode connected thereto. The lead body can include a main body portion extending between the distal end portion and a proximal end portion. The lead can be placed in a deployment device so that the prongs obtain a stored configuration in which the prongs are arranged substantially parallel to the main body portion of the lead. After placing the lead in the deployment device, a distal end portion of the deployment device can be positioned adjacent the target tissue. After positioning the distal end portion of the deployment device adjacent the target tissue, the distal end portion of the lead body can be deployed from the deployment device so that the prongs transition from the stored configuration to a deployed configuration and thereby at least partially wrap around the target tissue. After the prongs have wrapped around the target tissue, an electrical signal can be delivered to the at least one electrode to stimulate the target tissue.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a schematic illustration of a percutaneous, multi-pronged lead (in a deployed configuration) constructed in accordance with one aspect of the present disclosure;

FIG. 2 is a schematic illustration of the percutaneous, multi-pronged lead in FIG. 1 in a stored configuration;

FIG. 3 is a process flow diagram illustrating a method for applying stimulation to a target tissue of a subject according to another aspect of the present disclosure;

FIG. 4 is a schematic illustration showing the percutaneous, multi-pronged lead in FIG. 1 installed within a delivery device;

FIG. 5 is a schematic illustration showing the percutaneous, multi-pronged lead in FIG. 4 being deployed from the delivery device towards a target tissue;

FIG. 6 is a schematic illustration showing the percutaneous, multi-pronged lead in FIG. 5 grasping the target tissue; and

FIG. 7 is a schematic illustration showing the percutaneous, multi-pronged lead in FIG. 6 applying electrical stimulation to the target tissue.

### DETAILED DESCRIPTION

### Definitions

Unless defined otherwise, all technical and scientific terms used herein have the same meaning as is commonly understood by one of skill in the art to which the present disclosure pertains.

In the context of the present disclosure, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

As used herein, phrases such as “between X and Y” and “between about X and Y” can be interpreted to include X and Y.

As used herein, phrases such as “between about X and Y” can mean “between about X and about Y.”

As used herein, phrases such as “from about X to Y” can mean “from about X to about Y.”

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap (e.g., directly contact) or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms can encompass different orientations of the device in use or operation in addition to the orientation depicted in the figures. For example, if the device in the figures is inverted, elements described as “under” or “beneath” other elements or features would then be oriented “over” the other elements or features.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or figures unless specifically indicated otherwise.

As used herein, the term “subject” can be used interchangeably with the term “patient” and refer to any warm-blooded organism including, but not limited to, human beings, pigs, rats, mice, dogs, goats, sheep, horses, monkeys, apes, farm animals, livestock, rabbits, cattle, etc.

As used herein, the term “target tissue” can refer to a desired portion of biological tissue (e.g., nervous tissue) which can be grasped and/or stimulated.

As used herein, the term “autonomic nervous tissue” can refer to any tissues of the sympathetic nervous system (SNS) or the parasympathetic nervous system (PNS) including, but not limited to, neurons, axons, fibers, tracts, nerves, plexus, afferent plexus fibers, efferent plexus fibers, ganglia, pre-ganglionic fibers, post-ganglionic fibers, afferents, efferents, and combinations thereof.

As used herein, the term “sympathetic nervous tissue” can refer to any tissues of the SNS including, but not limited to, neurons, axons, fibers, tracts, nerves, plexus, afferent plexus fibers, efferent plexus fibers, ganglia, pre-ganglionic fibers, post-ganglionic fibers, cervical ganglia/ganglion, a cervicothoracic or stellate ganglion, thoracic ganglia/ganglion, afferents, efferents, and combinations thereof. In some instances, sympathetic nervous tissue can comprise a SNS nerve target. In some instances, the term can also refer to peripheral sympathetic nervous tissue.

As used herein, the terms “modulate” or “modulating” with reference to nervous tissue can refer to causing a change in neuronal activity, chemistry, and/or metabolism. The change can refer to an increase, decrease, or even a change in a pattern of neuronal activity. The terms may refer to either excitatory or inhibitory stimulation, or a combination thereof, and may be at least electrical, magnetic, optical or chemical, or a combination of two or more of these. The terms “modulate” or “modulating” can also be used to refer to a masking, altering, overriding, or restoring of nervous tissue activity.

As used herein, the term “in communication” can refer to at least a portion of a component, element, or structure being adjacent, in the general vicinity, in close proximity, and/or directly next to a second component, element, or structure.

As used herein, the term “electrical communication” can refer to the ability of an electric field generated by an electrode or electrode array to be transferred, or to have a neuromodulatory effect, within and/or on at least one nerve, neuron, and/or other nervous tissues (e.g., of the ANS).

As used herein, the terms “substantially blocked” or “substantially block” when used with reference to nervous tissue activity can refer to a complete (e.g., 100%) or partial inhibition (e.g., less than 100%, such as about 90%, about 80%, about 70%, about 60%, or less than about 50%) of nerve conduction through the nervous tissue.

As used herein, the term “activity” when used with reference to nervous tissue can, in some instances, refer to the ability of a nerve, neuron, or fiber to conduct, propagate, and/or generate an action potential. In other instances, the term can refer to the frequency at which a nerve or neuron is conducting, propagating, and/or generating one or more action potentials at a given moment in time. In further instances, the term can refer to the frequency at which a nerve or neuron is conducting, propagating, and/or generating one or more action potentials over a given period of time (e.g., seconds, minutes, hours, days, etc.).

### Overview

The present disclosure relates generally to devices and methods for neuromodulation and, more particularly, a percutaneous, multi-pronged lead and related methods for improved targeting and stimulation of nervous tissue. Conventional leads used for neuromodulation must be separately attached to a nerve target (e.g., using sutures), which requires complex surgery and creation of large incisions. Advantageously, the present disclosure provides electrical leads that can grasp a nerve target (e.g., autonomic or sympathetic nervous tissue) without requiring an attachment means (e.g., sutures), thereby reducing the invasive and complex nature of percutaneous neuromodulation procedures.

### Devices

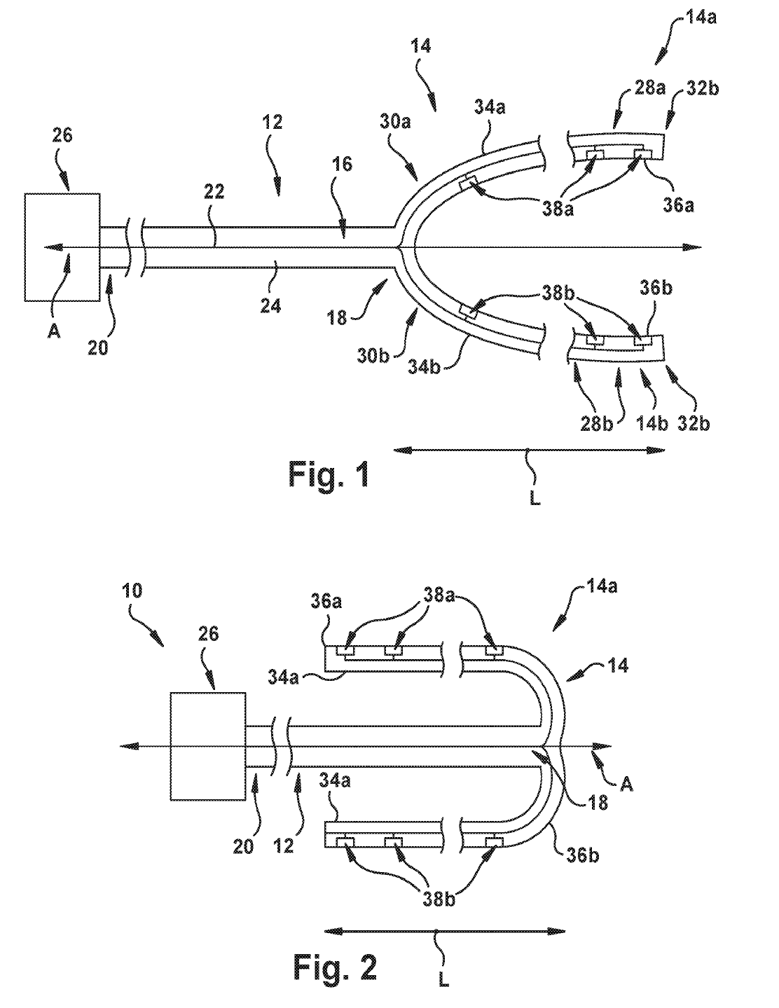

One aspect of the present disclosure can include a percutaneous multi-pronged lead 10 (FIGS. 1-2). The multi-pronged lead 10 can comprise a lead body 12 and one or more prongs 14 extending therefrom. The lead body 12 can include a main body portion 16 extending between a distal end portion 18 and a proximal end portion 20. The lead body 12 can be formed from one or more electrically-conductive elements 22 (e.g., wires) surrounded by an insulative layer 24. The electrically-conductive elements 22 can be made from one, or a combination of, electrically-conductive materials (e.g., copper, aluminum, etc.). The insulative layer 24 can be made from one or a combination of dielectric materials (e.g., plastics, ceramics, etc.) that resist the flow of electric charge. A portion of the lead body 12 can be operably connected to an external power source 26. For example, the proximal end portion 20 can be adapted for connection to the external power source 26 so that the electrically-conductive elements 22 are in electrical communication therewith. The lead body 12 can also have shape memory characteristics. In one example, a portion of the lead body 12 can be made from a shape memory material (e.g., nitinol). All or only a portion of the lead body 12 can have a rigid, semi-rigid, or flexible configuration.

In another aspect, one or more prongs 14 can extend from or beyond the distal end portion 18 of the lead body 12 when the lead 10 is in the deployed configuration. For the purpose of illustration only, the multi-pronged lead 10 will be shown and described as having first and second prongs 14a and 14b. It will be appreciated, however, that the multi-pronged lead 10 can include three, four, or more prongs 14. Advantageously, the first and second prongs 14a and 14b can be configured to transition from a stored configuration (FIG. 2) to a deployed configuration (FIG. 1) to at least partially, or entirely, wrap around, and apply stimulation to, a target tissue without the need for sutures or other attachment means.

Each of the first and second prongs 14a and 14b can include first and second prong bodies 28a and 28b having a proximal end 30a and 30b spaced apart from a distal end 32a and 32b. For example, the proximal ends 30a and 30b can be attached to the distal end portion 18 of the lead body 12. The first and second prong bodies 28a and 28b can each include a first surface 34a and 34b and an opposing second surface 36a and 36b. As shown in FIGS. 1-2, each of the prongs 14a and 14b can have an elongate, finger-like shape; however, other shapes are possible, such as circular (e.g., disc-shaped), square, triangular, frusto-conical, etc. The prongs 14a and 14b can have the same or different shapes depending, for example, upon the type and/or location of the target tissue. Similarly, the prongs 14a and 14b can have the same or different length L.

As shown in FIG. 1, the first and/or second prong(s) 14a and 14b can include one or more electrodes 38a and 38b connected thereto. In one example, each of the first and second prongs 14a and 14b can include electrodes 38a and 38b attached thereto (e.g., directly attached). The electrodes 38a and 38b can be located between the proximal ends 30a and 30b and the distal ends 32a and 32b of the first and second prong bodies 28a and 28b. The electrodes 38a and 38b can be spaced apart (e.g., equally) from one another along the first and second prong bodies 28a and 28b. In some instances, the electrodes 38a and 38b can be arranged on each prong 14a and 14b in the same or different configurations. In one example, the electrodes 38a and 38b can be grouped on one or more of the prongs 14a and 14b (e.g., in a cluster) in a particular pattern or geometry (e.g., a linear array). As shown in FIGS. 1-2, the electrodes 38a and 38b can be operably connected to the electrically-conductive elements 22. A portion of each of the electrodes 38a and 38b can be surrounded by, and embedded within, the insulative layer 24. In one example, a top surface of each electrode 38a and 38b can be exposed (e.g., not surrounded by or embedded within the insulative layer 24) to ensure that the electrode is in electrical communication with a target tissue.

The first and second prongs 14a and 14b can have a substantially C-shaped cross-sectional shape, or any other suitable cross-sectional shape (e.g., U-shaped, V-shaped, circular, rectangular, square, crescent, etc.) adapted to grasp a target tissue. The first and second prongs 14a and 14b can be formed from the electrically-conductive elements 22 and the insulative layer 24. The first and second prongs 14a and 14b can also be made from a shape memory material (e.g., Nitinol). All or only a portion of the first and second prongs 14a and 14b can have a rigid, semi-rigid or flexible configuration. For example, the first and second prongs 14a and 14b can have a flexible configuration to transition the first and second prongs from the stored configuration to the deployed configuration.

In the deployed configuration, the first and second prongs 14a and 14b can extend distally away from the distal end portion 18 of the lead body 12. In one example, each of the first and second prongs 14a and 14b, in the deployed configuration, can have a convex, concave, or substantially parallel configuration with respect to a longitudinal axis A of the lead body 12. In the stored configuration, the first and second prongs 14a and 14b can extend proximally away from the distal end portion 18 of the lead body 12. In one example, each of the first and second prongs 14a and 14b can have a convex, concave, or substantially parallel configuration with respect to the longitudinal axis A when the lead 10 is in the stored configuration. In some instances, the first and second prongs 14a and 14b can be disposed about opposing sides (e.g., 180° apart relative to the longitudinal axis A of the main body portion 16 while in the stored configuration.

In some instances, the first and second prongs 14a and 14b can transition between the deployed configuration and a non-deployed (e.g., stored) configuration by virtue of their partial or entire construction from a shape memory material. In other instances, the first and second prongs 14a and 14b can be associated with a securing mechanism (not shown) (e.g., clips, magnets, rubber bands, etc.) used to bias and hold the first and second prongs 14a and 14b in the non-deployed configuration. In one example, the securing mechanism can be located at the distal ends 32a and 32b of the first and second prongs 14a and 14b and then actuated (e.g., by severing, magnetic activation, etc.) to permit the first and second prongs 14a and 14b to transition into the deployed configuration.

### Methods

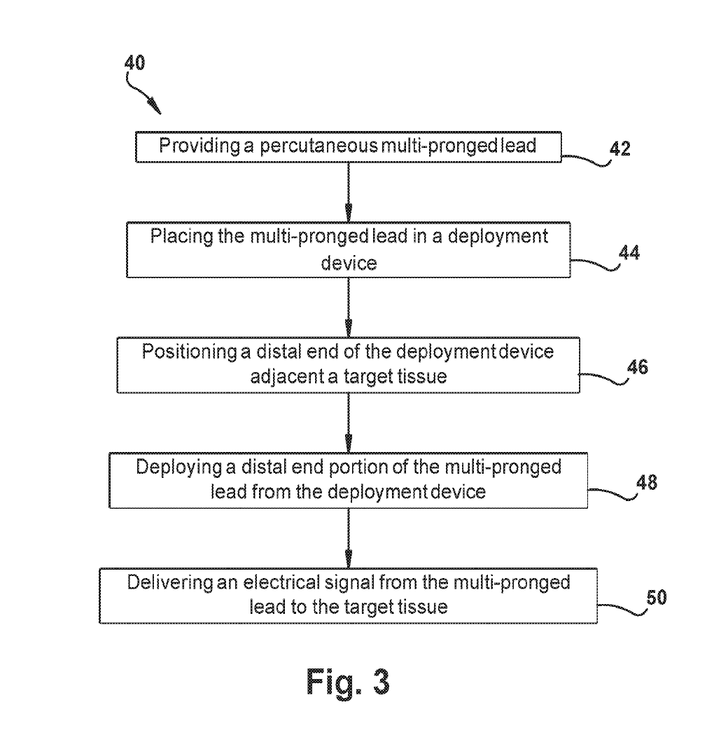

Another aspect of the present disclosure can include a method 40 (FIG. 3) for stimulating a target tissue in a subject. The method 40 can find use in a variety of percutaneous surgical or medical procedures where modulation (e.g., stimulation or blocking) of nervous tissue is desired. Non-limiting examples of nervous tissue whose activity can be modulated by the method 40 can include autonomic nervous tissue (e.g., sympathetic nervous tissue) and deep peripheral nervous tissue.

As shown in FIG. 3, the method 40 can generally comprise the steps of: providing a percutaneous multi-pronged lead (Step 42); placing the multi-pronged lead in a deployment device (Step 44); positioning a distal end of the deployment device adjacent a target tissue (Step 46); deploying a distal end portion of the multi-pronged lead from the deployment device (Step 48); and delivering an electrical signal from the multi-pronged lead to the target tissue (Step 50).

At Step 42, a percutaneous multi-pronged lead 10 can be provided. In some instances, the multi-pronged lead 10 can be configured as shown in FIG. 1 and described above. For example, the multi-pronged lead 10 can comprise a lead body 12 having first and second prongs 14a and 14b extending therefrom. One or more electrodes 38a or 38b can be disposed on a portion of the first or second prongs 14a or 14b. It will be appreciated that the particular dimensions of the multi-pronged lead 10 can depend upon the particular medical or surgical indication.

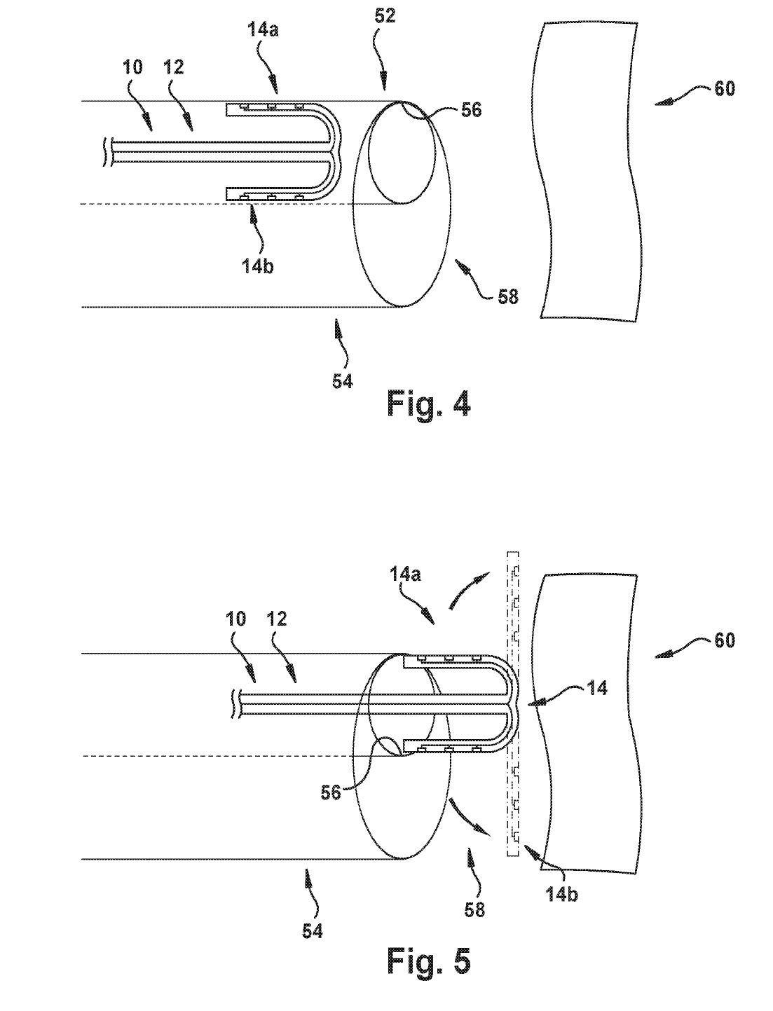

At Step 44, the multi-pronged lead 10 can be placed in a deployment device 52 (FIG. 4). In some instances, the deployment device 52 can be associated with a portion of an endoscope 54. For example, the deployment device 52 can comprise a working lumen of an endoscope 54. To place the multi-pronged lead 10 in the deployment device 52, the first and second prongs 14a and 14b can placed in the stored configuration by folding the prongs so that they are substantially parallel to the lead body 12. In the stored configuration, one or both of the prongs 14a and 14b may be in direct contact with the distal end portion 18 of the lead body 12. As shown in FIG. 4, the first and second prongs 14a and 14b are maintained in the stored configuration by virtue of contact made with the inner surface 56 of the deployment device 52.

At Step 46, a distal end 58 of the deployment device 52 can be positioned adjacent a target tissue 60 (e.g., an autonomic or sympathetic nervous tissue). For example, the deployment device 52 can be percutaneously inserted into the subject and then advanced towards the target tissue 60. In some instances, “adjacent a target tissue” can mean that all or only a portion of the distal end 58 is in direct contact with the target tissue. In other instances, “adjacent a target tissue” can mean

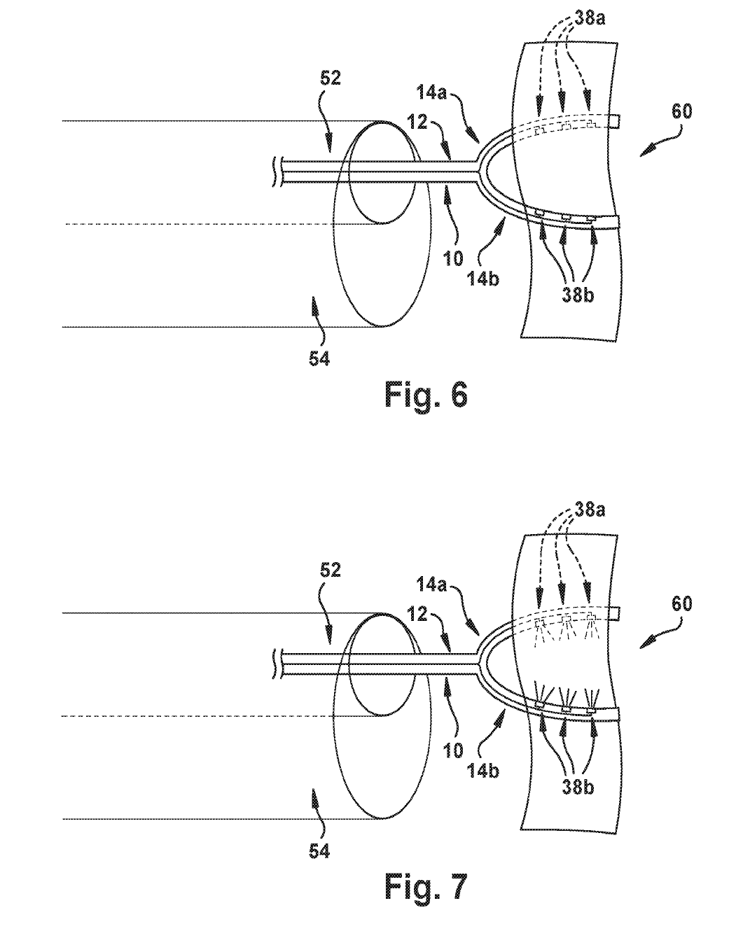

At Step 48, the distal end portion 18 of the lead body 12 can be deployed from the distal end 58 of the deployment device 52 (e.g., by urging the lead 10 in a distal direction) after positioning the distal end of the deployment device adjacent the target tissue 60 (FIG. 5). As the distal end portion 18 emerges from the deployment device 52, the first and second prongs 14a and 14b can freely (and automatically, in some instances, e.g., where the prongs are formed from a shape-memory material) transition from the stored configuration into the deployed configuration. In doing so, the first and second prongs 14a and 14b can at least partially or entirely wrap around the target tissue 60 (FIG. 6) (e.g., and thereby be in direct contact therewith) so that one or more of the electrodes 38a and 38b is placed into electrical communication with the target tissue. Advantageously, the wrap-around feature of the first and second prongs 14a and 14b eliminates the need to directly attach the multi-pronged lead 10 to the target tissue 60 using an attachment means (e.g., sutures), thereby reducing the invasive and complex nature of percutaneous neuromodulation procedures.

At Step 50, an electrical signal is delivered from the multi-pronged lead 10 to the target tissue 60 (FIG. 7). For example, an external power source 26 (not shown in FIG. 7) can be activated to deliver an electrical signal to the electrode(s) 38a and 38b via the electrically-conductive elements 22, thereby modulating activity of the target tissue 60.

From the above description of the present disclosure, those skilled in the art will perceive improvements, changes, and modifications. For example, it will be appreciated that the multi-pronged lead 10 can remain in the subject for chronic delivery of electrical signals to the target tissue 60 or, alternatively, that electrical signals can be delivered for a short (acute) period of time, whereafter the multi-pronged lead 10 can be removed from the subject. Such improvements, changes, and/or modifications are within the skill of the art and are intended to be covered by the appended claims.

1. A percutaneous, multi-pronged lead comprising:
a lead body having a multi-pronged distal end portion comprising a plurality of prongs, at least one of the prongs having an electrode connected thereto;
wherein each of the prongs is configured to transition from a stored configuration into a deployed configuration such that the prongs at least partially wrap around a target tissue.

2. The percutaneous, multi-pronged lead of claim 1, wherein the lead body includes a main body portion extending between the distal end portion and a proximal end portion, the prongs extending distally away from the main body portion when the prongs are in the deployed configuration.

3. The percutaneous, multi-pronged lead of claim 1, wherein the prongs are substantially parallel to the main body portion when the prongs are in the stored configuration.

4. The percutaneous, multi-pronged lead of claim 1, wherein the prongs are disposed on opposing sides of the main body portion when in the stored configuration.

5. The percutaneous, multi-pronged lead of claim 1, wherein the prongs are maintained in the stored configuration when the distal end portion is disposed in a deployment device.

6. The percutaneous, multi-pronged lead of claim 5, wherein the prongs transition to the deployed configuration when the distal end portion is no longer disposed in the deployment device.

7. A percutaneous, multi-pronged lead consisting of:
a lead body having a multi-pronged distal end portion comprising a plurality of prongs, at least one of the prongs having an electrode connected thereto;
wherein each of the prongs is configured to transition from a stored configuration into a deployed configuration such that the prongs at least partially wrap around a target tissue.

8. The percutaneous, multi-pronged lead of claim 7, wherein the lead body includes a main body portion extending between the distal end portion and a proximal end portion, the prongs extending distally away from the main body portion when the prongs are in the deployed configuration.

9. The percutaneous, multi-pronged lead of claim 7, wherein the prongs are substantially parallel to the main body portion when the prongs are in the stored configuration.

10. The percutaneous, multi-pronged lead of claim 7, wherein the prongs are disposed on opposing sides of the main body portion when in the stored configuration.

11. The percutaneous, multi-pronged lead of claim 7, wherein the prongs are maintained in the stored configuration when the distal end portion is disposed in a deployment device.

12. The percutaneous, multi-pronged lead of claim 11, wherein the prongs transition to the deployed configuration when the distal end portion is no longer disposed in the deployment device.

13. A method for stimulating a target tissue in a subject, the method comprising the steps of:
providing a multi-pronged lead comprising a lead body with a multi-pronged distal end portion comprising a plurality of prongs, at least one of the prongs having an electrode connected thereto, the lead body including a main body portion extending between the distal end portion and a proximal end portion;
placing the lead in a deployment device so that the prongs obtain a stored configuration in which the prongs are arranged substantially parallel to the main body portion of the lead;
positioning a distal end of the deployment device adjacent the target tissue;
deploying the distal end portion of the lead body from the deployment device so that the prongs transition from the stored configuration to a deployed configuration and thereby at least partially wrap around the target tissue; and
delivering an electrical signal to the at least one electrode to stimulate the target tissue.

14. The method of claim 13, wherein the step of placing the lead in the deployment device further comprises:
installing the lead within a lumen of the deployment device so that a surface of the lumen provides a reaction surface against which the prongs contact in the stored configuration.

15. The method of claim 13, wherein said deploying step further comprises exuding the distal end portion from the deployment device so that the prongs transition to the deployed configuration when the distal end portion is no longer disposed in the deployment device.

16. The method of claim 13, wherein, in said deploying step, the prongs extend distally away from the main body portion of the lead body when the prongs are in the deployed configuration.

17. A method for stimulating a target tissue in a subject, the method comprising the steps of:
providing a multi-pronged lead comprising a lead body with a multi-pronged distal end portion having a plurality of prongs, at least one of the prongs having an electrode connected thereto, the lead body including a main body portion extending between the distal end portion and a proximal end portion;
placing the lead in a deployment device so that the prongs obtain a stored configuration in which the prongs are arranged substantially parallel to the main body portion of the lead;
positioning, after the lead is placed in the deployment device, a distal end of the deployment device adjacent the target tissue;
deploying, after the distal end is positioned adjacent the target tissue, the distal end portion of the lead body from the deployment device so that the prongs transition from the stored configuration to a deployed configuration and thereby at least partially wrap around the target tissue; and
delivering, after the prongs have wrapped around the target tissue, an electrical signal to the at least one electrode to stimulate the target tissue.

18. The method of claim 17, wherein the step of placing the lead in a deployment device further comprises:
installing the lead within a lumen of the deployment device so that a surface of the lumen provides a reaction surface against which the prongs contact in the stored configuration.

19. The method of claim 17, wherein said deploying step further comprises exuding the distal end portion from the deployment device so that the prongs transition to the deployed configuration when the distal end portion is no longer disposed in the deployment device.

20. The method of claim 17, wherein, in said deploying step, the prongs extend distally away from a main body portion of lead body when the prongs are in the deployed configuration.
